(19) United States
(12) Patent Application Publication
(10) Pub. No.: US 2004/0185110 A1
(43) Pub. Date: Sep. 23, 2004

(54) Formulations of low solubility bioactive agents and processes for making the same

(76) Inventors: Ronald Harland, Yardley, PA (US); Chenkou Wei, Princeton Junction, NJ (US); Soojin Kim, West Orange, NJ (US); Alice Huey-Mei Hsieh, Edison, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/701,229

(22) Filed: Nov. 4, 2003

Related U.S. Application Data
(60) Provisional application No. 60/424,747, filed on Nov. 8, 2002.
(60) Provisional application No. 60/433,689, filed on Dec. 16, 2002.

(51) Int. Cl. A61K 9/14

(52) U.S. Cl. 424/489

(57) ABSTRACT

A method of coprocessing a limited solubility bioactive agent with a compatible aid comprising: (a) identifying a compatible aid for the bioactive agent; (b) either (i) forming a co-dissolved solution of the compatible aid and bioactive agent in a common solvent or (ii) forming a solution of the compatible aid in an anti-solvent and forming solution of the bioactive agent in a solvent; and (c) forming a film or primary particles from the co-dissolved solution or solutions of step (b), which film or primary particles comprise bioactive agent in crystalline form, with the crystals having average diameter of 1 micron or less.

This application claims a benefit of priority from U.S. Provisional Applications Nos. 60/424,747 and 60/433,689, the entire disclosure of which is herein incorporated by reference.

The present invention relates to biologically active compounds co-processed with one or more compatible materials to form particles which exhibit improved pharmaceutically important properties such as rate of dissolution and bioavailability, while providing the bioactive agent in a crystalline form. The primary particles contain embedded crystals of bioactive agent that are smaller than the primary particles. In one preferred embodiment, the invention relates to co-processed particles containing selective COX-2 inhibitors. The invention also relates to methods for processing the described biologically active compounds and one or more compatible materials. Also, in some embodiments, the invention provides a formulation that comprises relatively low concentrations of excipients compared to bioactive agent, providing a significant benefit by increasing the dissolution rate and/or enhancing the bioavailability of the bioactive agent when administered orally or through alternative routes such as buccal/sublingual, nasal, rectal, pulmonary or transdermal routes.

The prior art has suffered from the low level of bioavailability of certain bioactive agents. Limited solubility bioactive agents have been previously provided in solid dispersions. In most of the solid dispersion prior art, the bioactive agent exists in a predominantly amorphous state, which state has high energy and can therefore be unstable over longer time frames. Thus, for example, crystals may form or other phase transitions may occur, changing the dissolution and bioavailability characteristics of the formulation. The present invention addresses the need for increased stability of bioavailability-enhancing formulations.

Examples of the solid dispersion art that relies on amorphous bioactive agents can be found in Jung et al., Int'l J. Pharmaceuticals 187: 209-218, 1999, Kwon et al., WO 01/41765 and Wang et al., WO 01/85135. In Wang et al., WO 01/85135, for example, the need to use a disordered state is emphasized in the paragraph bridging pages 7 and 8. An example in Wang et al., WO 01/85135 uses Poloxomer™ polymer in a spray drying process, but the disclosure does not identify which of the many Poloxomer™ polymers is useful for these purposes and does not describe the crystallinity of the spray dried product.

Nonsteroidal anti-inflammatory drugs (NSAIDs) are widely prescribed for patients with rheumatic disease and pain. While they provide effective anti-inflammatory therapy and pain relief, a serious concern is the associated incidence of side effects, particularly gastrointestinal (GI) and renal side effects. Considering the huge number of users of NSAIDs on a worldwide basis, such concerns emphasize the need for new potent bioactive agents/drugs with improved tolerability.

The concept of different isoforms of COX was proposed in the mid 1970s by Vane and his colleagues, based on the fact that COX enzyme preparations from different tissues displayed different sensitivities to various NSAIDs. Concrete evidence of this hypothesis was only obtained in the 1990s when a second isoform of COX named COX-2 was discovered and a new hypothesis concerning the action of NSAIDs was proposed. The constitutive enzyme, COX-1, is thought to be a housekeeping enzyme playing a key role in the production of prostaglandins useful for physiological purposes such as gastric mucosa and kidney protection. The second isoform, COX-2, is inducible, is expressed in connection with inflammation or cell damage, and is responsible for the production of prostoglandins involved in the inflammation process.

Inhibition of COX-1 is thought to produce the undesirable side effects of NSAIDs whereas inhibition of COX-2 is responsible for analgesic and anti-inflammatory effects. Commonly used NSAIDs are active in inhibiting COX-2; and at the same time potent inhibitors of COX-1. By developing anti-inflammatory drugs that selectively target COX-2 inhibition, treatment of pain and inflammation can be at least as effective as that achieved with currently available NSAIDs, but is safer in terms of gastrointestinal and other common side effects. In this respect, clinical results obtained with two marketed COX-2 inhibitors (Celebrex™ and ViOXX™) have validated this concept. CELEBREX™ (celecoxib) is chemically designated as 4-[5-(4-methylphenyl)-3-(trifluoromethyl)-1H-pyrazol-1-yl]benzenesulfonamide and is a selective cyclooxygenase-2 (COX-2) inhibitor approved for the treatment of osteoarthritis and rheumatoid arthritis. See, e.g., U.S. Pat. Nos. 5,466,823 and 5,563,165, incorporated by reference herein in their entirety. VIOXX™ (rofecoxib) is chemically designated as 4-[4-(methylsulfonyl)phenyl]-3-phenyl-2(5H)-furanone and is a selective COX-2 inhibitor approved for the treatment of osteoarthritis, treatment of primary dysmenorrhea and management of acute pain. See e.g., U.S. Pat. No. 5,474,995, incorporated by reference herein in its entirety.

(Z)-3-[1-(4-bromophenyl)-1-(4-methylsulfonylphenyl) methylidine]-dihydrofuran-2-one (“Compound A”) and (Z)-3-[1-(4-chlorophenyl)-1-(4-methylsulfonylphenyl)methylidine] dihydrofuran-2-one are potent and selective inhibitors of COX-2, useful for the treatment of acute and chronic pain. See U.S. Pat. Nos. 5,807,873, 6,180,651 and related applications or patents, which are incorporated by reference herein in their entirety. Another such potent and selective inhibitor of COX-2 is 5-Chloro-3-(4-methanesulfonyl-phenyl)-6′-methyl-[2,3′]bipyridinyl, which is further described in WO 99/15503 (incorporated by reference herein in its entirety, but particularly pages 4-28). These and other COX-2 selective inhibitors falling within the biarylheterocycle genus or more particularly biarylfurnanone and biarylpyrazole genera appear to have low aqueous solubility thus suggesting suboptimal bioavailability. Compound A and other COX-2 inhibitors provide a class of compounds that are particularly preferred for formulation according to the invention. The bisarylheterocyclic genus, of which genus all the above-discussed COX-2 inhibitors are members, is a preferred class of COX-2 inhibitors.

A solid dispersion involves the formation of a eutectic mixture(s) of the drug with a carrier(s) and can be a means of formulating drugs with poor aqueous solubility. Solid dispersion approaches known in the art [1-8] were first developed by Sekiguchi and Obi in 1961 [1]. The drug in the solid dispersion is either in a microcrystalline state [1] or molecularly dispersed in the carrier [2-5]. Drug concentrations in most solid dispersions are relatively low, often less than 50% (wt/wt).

Methods have been discovered for processing low solubility bioactive agents with a compatible aid, to produce co-processed particles that have greater bioactive agent dissolution rate and or greater bioactive agent bioavailability as compared to the bioactive agent alone, a physical mixture of the bioactive agent with the compatible aid, or a formulation produced using conventional excipients and conventional manufacturing processes. In the co-processed particles described for the invention, the drug exists in a crystalline form, with the bioactive agent crystals preferably about 1 micron or less in size (preferably about 500 nm or less, more preferably 100 nm or less). The compatible aid can constitute 5 to 95% of the co-processed particles, but preferably constitutes less than 51% of the co-processed particles. The particles can be further formulated by conventional means. Methods of identifying compatible aids for a particular bioactive agent have been identified as part of the invention.

Compatible Aid or CA refers to a compound selected (typically by a screening method) for its ability to co-dissolve in a volatile solvent (or solvent mixture) with a given bioactive agent (to which agent it is a CA), at some ratio, such that when the solvent is vaporized a composition with improved dissolution (measured as described below) and containing crystals of bioactive drug is formed. The presence of crystals is determined by any appropriate method, including birefringence using hot-stage microscopy. In a preferred embodiment of the screening, the presence of crystals is determined using birefringence by hot-stage microscopy.

### SUMMARY OF THE INVENTION

The present invention addresses the above problems in the prior art by providing formulations of bioactive agent in crystalline form that have relatively high bioavailability and relatively high loading of bioactive agent. As such the present invention is, in one embodiment, a formulation of a bioactive agent co-processed with a CA to form particles in which the bioactive agent is in crystalline form. These particles can have increased dissolution rate or bioavailability as compared with the bioactive agent alone or formulated with conventional excipients using conventional processes such as direct compression or dry or wet granulation, or a physical mixture of bioactive agents and the CA. These conventional excipients and processes are known to those skilled in the art and can be found for example in Remington's Pharmaceutical Sciences, 20th edition, 2000 (incorporated by reference in relevant part), a standard reference in the field. Further, the present invention provides methods for processing the formulation. Thus, in one embodiment the invention provides a method of coprocessing a limited solubility bioactive agent with a compatible aid comprising: (a) identifying a compatible aid for the bioactive agent; (b) either (i) forming a co-dissolved solution of the compatible aid and bioactive agent in a common solvent or (ii) forming a solution of the compatible aid in an anti-solvent and forming a solution of the bioactive agent in a solvent; and (c) forming a film or primary particles from the co-dissolved solution or solutions of step (b) (for which the primary particles are preferably of average diameter of 15 microns or less, or 10 microns or less, or 5 microns or less, or 2 microns or less), and which film or primary particles comprise bioactive agent in crystalline form, with the crystals having average diameter of 1 micron or less.

The forming process can be:

(i) spray drying the co-dissolved solution to remove the solvent, or

(ii) (1) mixing the co-dissolved solution with an antisolvent for the bioactive agent using impinging jets, or

(ii)(2) mixing the bioactive agent solution with the solution of compatible aid in antisolvent using impinging jets, or

(iii) conducting process (ii)(1) or (ii)(2) and drying the product by spray drying, or

(iv) batch precipitation of the co-dissolved solution or batch precipitation of the solution of drug with the compatible aid in antisolvent.

The process is selected to provide, as facilitated by the selection of the CA, particles or films that exhibit faster bioactive agent dissolution, or greater bioactive agent bioavailability, or have faster onset. Faster dissolution or greater bioavailability are more often the sought-after properties. Particles are a preferred product of the process. Particularly preferred processes are processes (i), (ii), (iii) and (iv).

### BRIEF DESCRIPTION OF THE DRAWINGS

FIG. 1 is a diagram of a spray drying apparatus.

FIG. 2 is a diagram of an impinging jet apparatus.

FIG. 3 shows X-ray diffraction patterns for formulations of Compound A.

FIG. 4 shows dissolution profiles for formulations of Compound A.

FIG. 5 shows a scanning electron microscopy image of a co-processed formulation of Compound A.

FIG. 6 compares particles of the invention versus micronized powder using hot-stage microscopy.

FIG. 7 shows pharmacokinetic profiles.

### DEFINITIONS

A-bioactive agent or bioagent is a substance such as a chemical that can act on a cell, virus, tissue, organ or organism, including but not limited to insecticides or drugs (i.e., pharmaceuticals) to create a change in the functioning of the cell, virus, organ or organism. A limited solubility bioactive agent is one whose dissolution profile in aqueous solutions is such that one of skill in the art would recognize its solubility as restricting its bioavailability.

The comparison composition is bioactive agent of average diameter ˜5 microns that is physically mixed with the CA or a solid dosage form containing such bioactive agent and conventional excipients and prepared using conventional processes. Such a comparison composition can comprise micronized powder alone or suspensions of the bioactive agent.

As used herein co-processed particle may be in the form of a particle or agglomerate.

COX-2 selective inhibitors comprise a genus of organic compounds or pharmaceutically acceptable salts or solvates thereof which are each capable of selectively inhibiting the COX-2 enzyme over the COX-1 enzyme. The scope of the present invention additionally includes COX-2 inhibitors that are not selective over the COX-1 enzyme.

A composition of bioactive agent is in crystalline form if at least about 50% of the bioactive agent in the composition is crystalline, as measured by the method described below. In preferred embodiments of the invention, the bioactive agent is about 60% or more, or about 70% or more crystalline.

Faster Dissolution of a bioactive agent is measured in aqueous media that can contain surfactant using USP 1 or 2 and compared with a comparison composition. The aqueous medium is selected to discriminate between different compositions.

Faster Onset is measured in an animal (which is typically selected for being a member of a species that provides an appropriate animal model for the indication to be treated with the respective bioactive agent) or in humans, by comparison with the bioactive agent alone or a conventional formulation of the bioactive agent of average diameter ˜5 micron filled into capsules, pressed into tablets, or dosed as an aqueous suspension in, for example, methylcellulose with or without polysorbate (Tween) 80, or a physical mixture of the bioactive agent and the CA. Plasma levels are measured after each treatment as a function of time. A lower Tₘₐₓ indicates faster onset.

Relative oral bioavailability is measured in an animal (which is typically selected for being a member of a species that provides an appropriate animal model for the indication to be treated with the respective bioactive agent) or in humans by comparison with the bioactive agent alone or an oral solution of the bioactive agent or a conventional formulation of the bioactive agent of average diameter ˜5 microns filled into capsules, pressed into tablets, or dosed as an aqueous suspension in, for example, methylcellulose with or without polysorbate (Tween) 80, or a physical mixture of the bioactive agent and the CA. Plasma levels are measured after each treatment as a function of time. Relative bioavailability of the co-processed material and that of the capsule formulation of the bioactive agent is determined by calculating the area under the curve (AUC) after each treatment and divided by the AUC of the reference (oral solution).

### DETAILED DESCRIPTION OF THE INVENTION

The present invention provides co-processed particles comprising one or more compatible aids and a bioactive agent, particularly an agent of limited solubility such that the bioactive agent is in crystalline form within the co-processed material.

Bioactive agents suitable for use in the present invention include but are not limited to anabolic agents, antacid agents, analgesics, alkaloids, antiinflammatory agents, antiallergic agents, anti-Alzheimer's agents, antianginal agents, antianxiety agents, antiarrhythmic agents, antiarthritics, antiasthmatics, antibiotics, anticancer agents, anticholesterolaemics, anticoagulants, anticonvulsants, antidepressants, antidiabetic agents, antidiarrhoel preparations, antiemetics, antiepileptics, antifungals, antihelminthics, antihistamines, antihypertensives, antiinfectives, antilipid agents, antimanics, antimicrobials, antimuscarinic agents, antimycobacterials, antinauseants, antineoplastic agents, antiobesity agents, antiparasitics, antipsoriatics, antipsychotics, antipyretics, antischizophrenics, antispasmodics, antithrombotic agents, antithyroid agents, antitumor agents, antitussives, antiulceratives, antiurecemic agents, antivirals, anxiolytic sedatives, appetite suppressants, astringents, beta adrenoceptor blocking agents, bronchodilators, cerebral dilators, cardiovascular agents, central nervous system depressants and stimulants, cholesterol lowering agents, coronary dilators, contrast media, corticosteroids, cough suppressants, decongestants, diagnostic agents, diuretics, dopaminergics, erythropoietic agents, expectorants, gastrointestinal agents, hemostatics, hormonals, hyper and hypo glycemic agents, hypnotics, immunological agents, immunosuppressants, laxatives, lipid regulating agents, migraine treatments, mineral supplements, mucolytics, muscle relaxants, neuromuscular agents, oligonucleotides, parasympathomimetics, parathyroid calcitonin, peripheral vasodilators, peptides, prostaglandins, proteins, proton pump inhibitors, psycho-tropics, radio-pharmaceuticals, sedatives, sex hormones, steroids, stimulants, sympathomimetics, thrombolytics, thyroid agents, tranquilizers, uterine relaxants, vasoconstrictors, vasodilators, vitamins and xanthines and mixtures thereof.

The solubility in aqueous solution of the bioactive agents processed in the present invention is preferably less than about 10 mg/mL more preferably less than 1 mg/mL and most preferably less than about 0.1 mg/mL in water, 0.1 N HCl or over a pH range of 1-7. Preferred bioactive compounds include selective COX-2 inhibitors of the bisarylheterocyclic genus. One such embodiment of the present invention relates to co-processed particles incorporating a bisarylheterocyclic compound such as (Z)-3-[1-(4-bromophenyl)-1-(4-methylsulfonylphenyl) methylidine]-dihydrofuran-2-one or alternately 3-[1-(4-chlorophenyl)-1-(4-methylsulfonylphenyl) methylidine]-dihydrofuran-2-one and a polymer.

CAs include but are not limited to dissimilar bioactive compounds, polymers, pharmaceutical excipients, extracts and other natural materials, materials containing hydrophilic segments, surfactants, surface active agents, hydrogels, biomaterials, gums, peptides, celluloses, cellulosic derivatives, starches, lecithins, saccharides, polysaccharides, polyols, alcohols, hydrogenated materials, long chain acids and bases, esters, ethers, fatty acids, fatty alcohols, glycerides, waxes, oils, fats, high intensity or artificial sweeteners, vitamins, food and food ingredients, materials of biological origin, synthesized materials, and mixtures and derivatives thereof. The CA used to produce the particles is preferably a water dispersible polymer. More preferably the CA is a water soluble polymer. One class of such polymers are poloxamer polyols (also known as polyalkylene block copolymers). A preferred example is a Pluronic™ polymer.

Pluronic™ polymers are block copolymers of propylene oxide and ethylene oxide, and are generally surface active agents. Preferred Pluronic™ polymers are block copolymers of propylene oxide linearly sandwiched between ethylene oxides. Pluronic™ polymers with a melting point of greater than 35 degrees Celsius are preferred. A most preferred example of a Pluronic™ polymer is Pluronic™ F127 polymer.

In a preferred embodiment of the present invention the resultant primary particles are 15 microns or less, 10 microns or less, or 5 microns or less, or 2 microns or less, in diameter.

In some embodiments the present invention relates to co-processed particles incorporating a bioactive agent and a compatible aid such that the co-processed particles contain approximately from 5 to 95% wt of the bioactive agent and approximately from 5 to 95% wt of the compatible aid. In a preferred embodiment the co-processed particles incorporate a bioactive agent and a compatible aid such that the co-processed particles contain approximately from 20 to 60% wt of the bioactive agent and approximately from 40 to 80% wt of the compatible aid. In the most preferred embodiment the co-processed particles incorporate a bioactive agent and a compatible aid such that the co-processed particles contain approximately from 40 to 60% wt of the bioactive agent and approximately from 40 to 60% wt of the compatible aid.

While in one embodiment components of the formulation are a bioactive agent and a compatible aid, other components including conventional excipients can be present provided useful dissolution profiles are obtained.

The process of forming primary particles of the present invention may be achieved using conventional processes such as heating, cooling, evaporation, chemical reaction and changing solvent composition by using antisolvents to reduce the solubility of the bioactive agent and the CA. In certain preferred embodiments spray drying or use of impinging jets is employed.

Among preferred COX-2 inhibitors are those according to formula I:

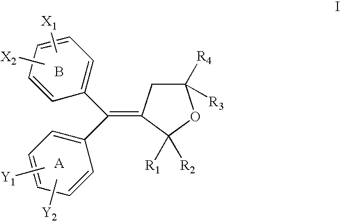

in which:

the rings A and B independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents X₁, X₂, Y₁ or Y₂ is necessarily:

an—S(O)ₙ—R group, in which n is an integer equal to 0, 1 or 2 and R is a lower alkyl radical having 1 to 6 carbon atoms or a lower haloalkyl radical having 1 to 6 carbon atoms, or

an—SO₂—NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

X₁ and X₂ or Y₁ and Y₂ are a methylenedioxy group; and

R₁, R₂, R₃ and R₄ independently are:

a hydrogen atom,

a halogen atom,

a lower alkyl radical having 1 to 6 carbon atoms,

a lower haloalkyl radical having I to 6 carbon atoms, or

an aromatic radical selected from the group consisting of phenyl, naphthyl, thienyl, furyl and pyridyl; or

R₁, R₂ or R₃, R₄ are an oxygen atom, or

R₁, R₂ or R₃, R₄ together with the carbon atom to which they are attached, form a saturated hydrocarbon ring having from 3 to 7 carbon atoms.

In some embodiments, the COX-2 inhibitors are those according to the formula II:

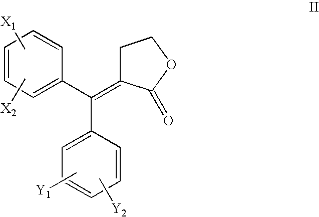

wherein X₁, X₂, Y₁ and Y₂ are as described above.

In the description and the claims, lower alkyl is understood as meaning a linear or branched hydrocarbon chain having from 1 to 6 carbon atoms. A lower alkyl radical is for example a methyl, ethyl, propyl, isopropyl, butyl, isobutyl, tert-butyl, pentyl, isopentyl, hexyl or isohexyl radical.

Lower haloalkyl radical is understood as meaning an alkyl radical having 1 to 6 carbon atoms in which 1 to 7 hydrogen atoms have been substituted by 1 to 7 halogen atoms. A lower haloalkyl radical is for example a trifluoromethyl radical, a 2,2,2-trifluoroethyl radical, a pentafluoroethyl radical, a 2,2,3,3,3-pentafluoropropyl radical, a heptafluoropropyl radical or a chloromethyl or bromomethyl radical.

Halogen is understood as meaning a chlorine, bromine, iodine or fluorine atom.

Saturated hydrocarbon ring having from 3 to 7 carbon atoms is understood as meaning cyclopropane, cyclobutane, cyclopentane, cyclohexane or cycloheptane.

Radical derived from a heterocycle means any aromatic ring containing from one to four heteroatoms in its ring: nitrogen, oxygen or sulfur.

Amongst these rings, pyridine, furan, thiophene, as well as pyrrole, irnidazole, pyrazole, pyrazine, pyrimidine, pyridazine, oxazole, oxadiazole, thiazole and thiadiazole are particularly preferred.

These COX-2 inhibitors are described in more detail in U.S. Pat. No. 5,807,873, which is incorporated herein by reference in its entirety.

FIG. 4 compares the dissolution profile of co-processed particles of the drug and Pluronic™ F127 polymer in varying ratios, and micronized (Z)-3-[1-(4-bromophenyl)-1-(4-methylsulfonylphenyl)methylidine] dihydrofuran-2-one in physical admixture with Pluronic™ F127 polymer. The dissolution profile is at the most preferred of those tested when the drug:Pluronic™ ratio is 50:50 by weight (Squares). At a drug:Pluronic™ ratio of 90:10 (Triangles), the dissolution is less than at the 50:50 ratio. The dissolution was conducted with 50 mg dosages of the drug in 50 mM sodium acetate, 3% sodium lauryl sulfate, pH 4.6, operating a paddle at 75 rpm. The control is a physical mixture of micronized drug mixed with Pluronic™ at 50:50 (Circles). (FIG. 4: Dissolution of Compound A (COX-2 inhibitor) spray-dried with Pluronic F127 vs a physical mixture. The dissolution of 50 mg of drug equivalent/capsule was monitored as a function of time in 50 mM Na acetate buffer, pH 4.6, with 3% SLS; paddles speed was 75 rpm. Squares: 50:50 Drug:Pluronic F127, co-Processed; Triangles: 90:10 Drug:Pluronic F27, co-Processed; Circles: 50:50 Physical Mixture of Drug and Pluronic F127.)

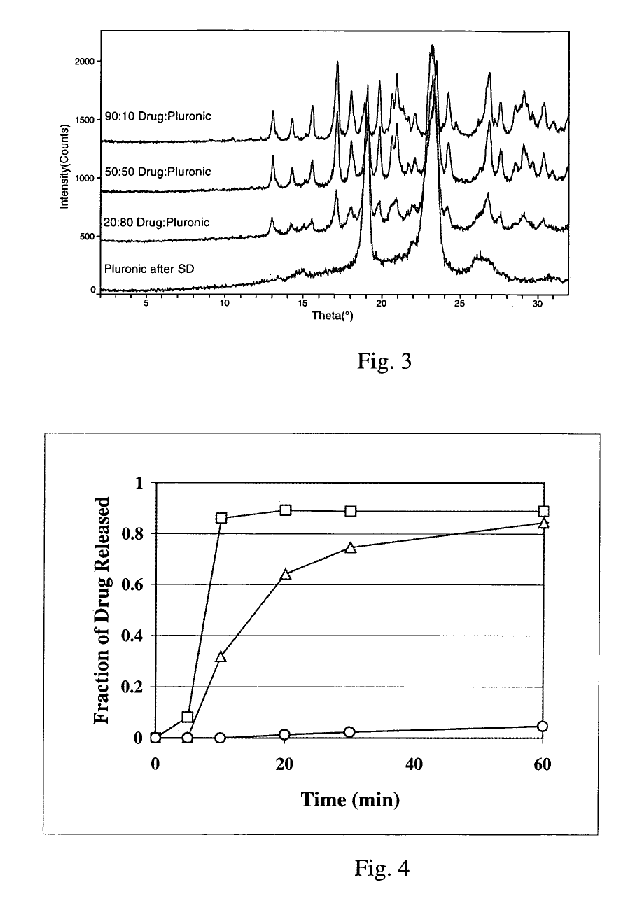

FIG. 3 shows the X-ray diffraction patterns for these formulations compared to processed Pluronic™. The data shows that the co-processed particles contain the drug in crystalline form.

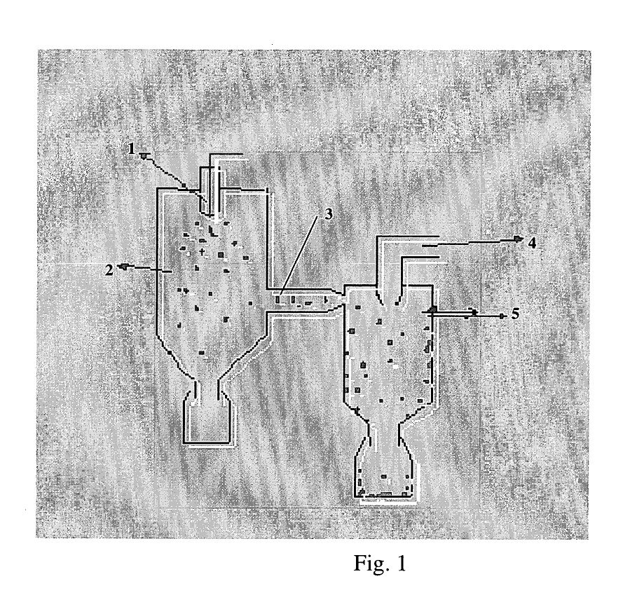

The spray-dry process can comprise combining a bioactive agent with a compatible aid in a common solvent system and drying the combination by evaporating the solvent while spray drying the combination; or using a process as described further in the examples herein. The resulting particles contain the bioactive agent in crystalline form. Conditions used for spray-drying, such as temperature and atomization air flow rate may vary according to the volatility of the solvent used, the initial concentration of drug and the compatible aid in the solvent, and chemical and physical properties of the drug and the compatible aid used. FIG. 1 diagrams a spray drying apparatus. The solution to be spray-dried and an atomization gas are injected into the drying chamber 2 through the injection port 1. The solution is dried and flows through conduit 3 into the collection chamber 5 where the solvent gas escapes through vent 4 and the co-processed particles are collected.

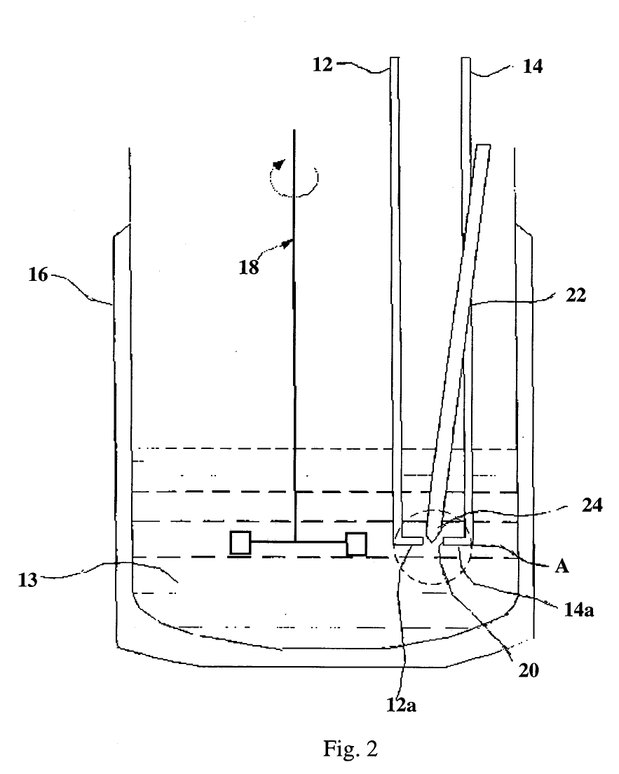

FIG. 2 illustrates an impinging jet apparatus described more fully in U.S. Pat. No. 6,302,958, which has a first jet 12 and a second jet 14 arranged substantially diametrically opposite one another in a flask 16, such as a 1000 ml flask, which is agitated by a overhead stirrer 18. Flask 16 contains bulk or liquid 13, which is advantageously the same material as that coming through second jet 14 (anti-solvent). First jet 12 and second jet 14 are provided with jet orifices 12a and 14a respectively, which are positioned substantially 180 degrees from each other at, for example, a distance of 0.4 inches from one another. The space 20 defined between first and second jet orifices 12a and 14a defines an impingement point where the fluid from first jet 12 and the fluid from second jet 14 impinge and micromix within flask 16.

A sonication probe or sonicator 22, such as a 20 khz sonication probe, having a probe tip 24 on one end, is positioned in flask 16. Probe tip 24 of a sonication probe 22 can be immersed in the crystallization slurry throughout the crystallization process. Probe tip 24 of sonicator 22 can be advantageously located as close as possible to the impinging point 20. Depending on several processing parameters such as temperature, liquid viscosity and percent solids, among others, probe 24 may provide up to 500 watts of power within the crystallization slurry. The addition of ultrasonic energy in the immediate vicinity of the impinging jets 12, 14 produces an average particle size of less than 1 micron.

Liquid can be pumped through first and second jets 12, 14 at a minimum linear velocity of 12 m/s. The liquid is comprised of one or more solvents which may include a combination of the pharmaceutical compound and a solvent and an anti-solvent, or simply a combination of solvents and an anti-solvent. When the two jet streams emerge and meet midway between orifice tips 12a, 14a, high intensity micromixing occurs and a disk of crystallization slurry is formed.

Batch precipitation methods include standard mixing of solvent and antisolvent, and changes in temperature to create a supersaturated state.

Results show that the use, for example, of Pluronic™ F127 polymer resulted in significantly improved dissolution rates compared to bioactive agent alone or a physical mixture of the bioactive agent and compatible aid in the same ratio. Increasing the ratio between the Pluronic™ polymer and the drug in spray dried particles led to an increase in dissolution rate. Particle size analysis shows that co-processed particles are in general larger than those of Compound A spray-dried without excipients. Thus the increase in dissolution rate cannot be explained by an increase in surface area. However, hot-stage microscopy shows that the bioactive agent crystals in the co-processed particles are much smaller than the unprocessed micronized bioactive agent. The increase in dissolution rate can therefore be attributed to the incorporation of the Pluronic™ polymer as a co-processing excipient and this co-processing leads to formation of a matrix of nanosized drug substance and the CA.

In an embodiment of the invention, a CA is selected by preparing a film as follows: a solution of 20:80 drug: excipient (e.g., 30 mg:120 mg in 1-10 mL of solvent), or 50:50 (e.g. 2.6 mg:2.6 mg in ˜0.5 mL solvent) or a similar ratio is prepared by dissolving the drug and excipient in a suitable solvent. The solvent can be organic or aqueous. The solution is allowed to evaporate in a suitable pan. The resultant film is either removed from the pan, or the film in the pan is used for evaluation.

The films or particles may be evaluated for any of the following, depending on the desired outcome: dissolution, crystallinity of the drug (estimated by powder x-ray diffraction (PXRD)), and microscopy, hot-stage microscopy, and high pressure liquid chromatography (HPLC) for potency and stability. For dissolution, the films can be removed from the pan and transferred into a capsule, or if the film is formed in a small pan, the entire pan can be placed in the dissolution vessel. Likewise for differential scanning calorimetry (DSC), samples of the film or the film in the pan contents can be evaluated. For PXRD, it is possible to prepare the film in the sample holder for direct evaluation. The results are compared to a film of drug prepared from the same solvent without the compatible aid, or a physical mixture of drug and compatible aid, and those films which show good comparative performance are considered for further evaluation.

A TA Instruments 2910 DSC instrument can be used. Bioactive agent, materials to be screened, and the cast blends are accurately weighed (˜5 mg) into sealed DSC aluminum pans. The samples are heated at 10 degrees C./min from ambient to a final of 250 degrees C. in a nitrogen atmosphere. A thermogram is recorded as a function of temperature to determine the melting point (T_{f}) and the heat of fusion (ΔH_{fus}).

Powder X-ray diffraction measurement on bioactive agent and co-processed particles can be obtained with a Philips ADP 3720 XRD using copper radiation with generator setting of 45 kV and 40 mA. Each sample is, for example, scanned between 2 and 32 degree 2θ and in step sizes of 0.04 degree 2θ.

Disintegration can be measured by placing 10 mg of bioactive agent equivalent of co-processed particles in 100 ml of water. The fluid is, for example, contained in a 150 ml beaker with rapid agitation provided by a rotating stir bar. The mixture is stirred for 30 minutes and visually examined.

The present invention addresses the prior art issues of low bioavailability of certain drugs such as COX-2 selective inhibitors useful for the treatment of, for example, arthritis and rheumatic pain. Co-processing the COX-2 selective inhibitor with a water soluble polymer excipient such as a Pluronic(TM) polymer has been shown to increase the dissolution rate and increase the bioavailability of the drug. Compound A, a COX-2 selective inhibitor, is a white to off-white odorless crystalline, anhydrous powder. At room temperature it is soluble in methylene chloride, and acetonitrile. The drug is poorly soluble in water and has neither acidic nor basic functions. The aqueous solubility of the drug is less than 2 82 g/mL at 22 degrees Celsius at pH 6.2.

In some embodiments the present invention relates to methods for co-processing a COX-2 selective inhibitor. The methods include forming co-processed microparticles by dissolving a CA and a COX-2 inhibitor in a volatile solvent to create a solution and spray drying the solution to form mlicroparticles. The volatile solvent can be selected from the group comprising methylene chloride, acetone, ethanol, chloroform, methanol and isopropanol, and other solvents that can be identified by those of skill in the art with reference to the solubility of the relevant CA and a COX-2 inhibitor.

Screening Techniques

The initial screening technique for identifying CAs visually examined films of the relevant bioactive agent and the prospective CA, prepared by vaporizing a common solvent. Visually homogeneous films that contained, on microscopic or spectroscopic examination, crystals of bioactive agent (preferably, about 20% or more), were deemed CAs. The visual homogeneity provided an indication that phase separation events would not disrupt the content uniformity of a pharmaceutical processed with the CA.

A preferred process is automated and operates in small volume (e.g., 10 microliters). Examination is for improved dissolution, and optionally, for evidence of a crystalline form of the bioactive agent, preferably greater than 10% crystallinity. Optionally, the materials can be tested for greater crystal content, such as 20, 30 or 40%. The screening technique does not have to achieve a crystalline form for the bioactive agent, since the processing of the invention can result in higher crystallinity than observed in the evaporative screening process.

Measuring Crystallinity

Crystal content can be assessed mathematically by adding portions of observed PXRD patterns of mock processed (by the invention) polymer and unprocessed pure crystalline bioactive agent to generate simulated “pattern I.” Pattern I was then fitted to an observed pattern of co-processed material derived by adding a small portion of amorphous bioactive agent to generate simulated “pattern II.” The percentages of these three components (namely bioactive agent, polymer and amorphous) were calculated based on the second step of simulation. The calculated amorphous amount should only be contributed by the amorphous amount derived from what would be crystalline bioactive agent in a purely crystal form. In an example using co-processed Compound A, the calculation showed that about 10 to 15% of drug substance was amorphous.

The size of the crystals of bioactive agent in a composition is measured by hot stage microscopy, with temperature used to melt the polymer. While it is theoretically possible for a small amount of crystal to dissolve in the polymer melt, the value so obtained is believed to be roughly accurate, and nonetheless provides the measurement used in with respect to this invention. In those instances where the heat-induced solubility of the bioactive agent in the polymer renders the hot-stage microscopy method ineffective, X-ray peak profile analysis or transmission electron microscopy can be used to measure the size of the crystals of bioactive agent.

Films

In one embodiment of the invention, films are formed by evaporating a co-solvent from the CA and bioactive agent.

PXRD analysis of co-processed Compound A and Pluronic F127 materials at ratios of 20:80, 50:50, and 90:10 bioactive agent:polymer are shown in FIG. 3 in comparison to pure polymer. The PXRD data indicates that the bioactive agent is in a crystalline form in the co-processed materials. Based on PXRD peak widths and hot-stage microscopy, the crystal size of the drug in the co-processed particles was found to be predominantly sub-micron in size.

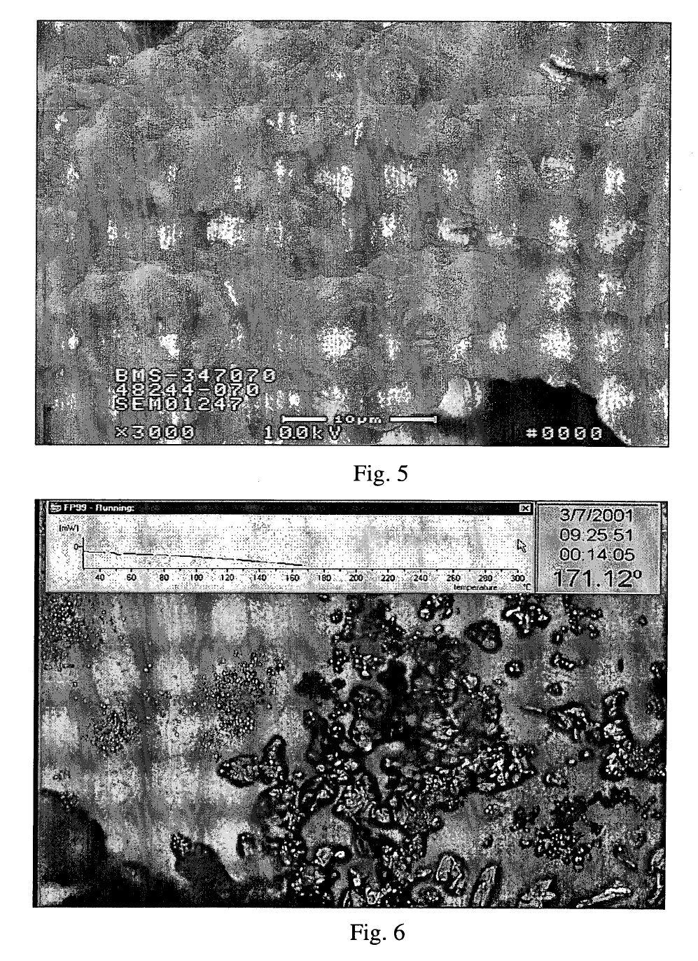

FIG. 5 shows the co-processed particles as seen by Scanning Electron Microscopy (SEM). (FIG. 5: SEM Image of Compound A spray-dried with Pluronic F127 in a 50:50 ratio.) FIG. 6 shows that the bioactive agent crystals, after melting the CA, exists predominantly in the sub-micron particle size range, as compared to the starting material in the micron-size range. (FIG. 6: Left: Compound A spray-dried with Pluronic F127, after Pluronic melted; Right: Micronized drug substance.)

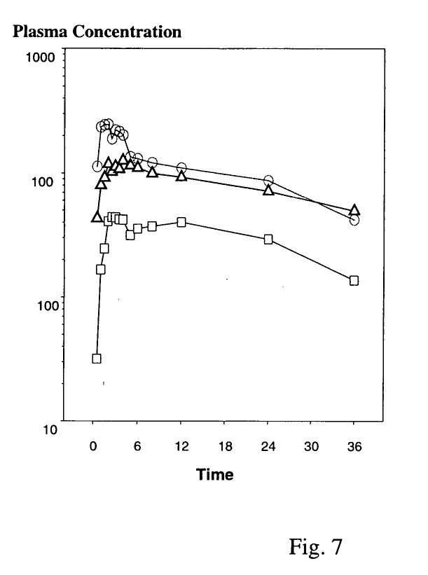

Bioavailability studies were performed in dogs using a solution of the bioactive agent, 50:50 bioactive agent:polymer co-processed material, and a formulation of the micronized drug filled into capsules. The dog plasma concentration vs. time profiles are shown in FIG. 8. As compared to the solution of the bioactive agent, the 50:50 bioactive agent:polymer co-processed material resulted in relative oral bioavailability of 70.2%, while the micronized bioactive agent formulation resulted in only 29% relative oral bioavailability. (FIG. 7: Pharmacokinetic profiles (ng/mL) obtained in dogs (N=3) after oral administration of 50 mg of drug equivalent/capsule. Circles: solution; Triangles: 50:50 Drug:Pluronic F127 co-processed by spray-drying; Squares: bulk drug.)

Powder X-ray Diffraction Measurement was obtained on the drug and the processed particles using a Philips MDP Xpert Powder X-Ray Diffraction System with copper radiation and a generator setting of 45 kV and 40 mA. Each sample was scanned between 2 and 32 degree 2Θ and in step sizes of 0.03 degree 2Θ.

Particle size was determined by depositing the particles onto a microscope slide using air stream dispersion. A magnification of 50× was used for analysis. Data was collected from 600 particles from the sample to ensure correct statistics. The particle sizes were calibrated by means of a stage micrometer.

For dissolution, particles were weighed into appropriately sized gelatin capsules and dissolution of the capsules was performed in a USP Apparatus 2. The medium was 50 mM acetate buffer and 3% sodium lauryl sulfate at a pH of 4.6. The medium volume was 1000 mL and the medium temperature maintained at 37 degrees Celsius. The agitation rate was 75 rpm and the samples were analyzed using HPLC.

### EXAMPLE 1

### Spray Drying Process

Solutions containing 5% (wt/wt) Compound A and the compatible aid (50:50 ratio) in methylene chloride or acetone were sprayed in a Buchi B-191 laboratory scale mini spray dryer using air or N2 gas. The following processing conditions which were used: inlet temperature setting of 34-35 C, aspirator setting at 100%, pump setting between 5 and 10%, flow control setting of 700. Instrument responses were outlet temperature between 23-25 C, back pressure of 35-40 mbar, and N2 or air pressure of 90 psi. Product was collected from the collection container and cyclone walls.

### EXAMPLE 2

### Impinging Jet Process

Dimethyl sulfoxide was used as organic solvent and water served as anti-solvent. An impinging jet (IJ) apparatus equipped with a sonication probe was used. An organic solution containing Compound A and the compatible aid was pumped through one jet and an aqueous phase was pumped through the other jet. In some operations, the compatible aid was dissolved in the aqueous phase in cases where its solubility in the organic solvent was low. The two liquid streams met at the IJ vessel that was maintained at 2 degrees C. The water acted as an anti-solvent to crystallize the drug along with the compatible aid. The suspension in IJ vessel was then filtered, washed and dried to obtain the final product.

### References

1. Sekiguchi, K., Obi, N. Chem. Pharm. Bull. 1961, 9, 866.

2. Goldberg, A. H., Gibaldi, M., and Kanig, J. L., J. Pharm. Sci., 1966, 55, 482.

3. Goldberg, A. H., Gibaldi, M., J. Pharm. Sci. 1966, 55, 487.

4. Serajuddin, A. T. M., J. Pharm. Sci., 1999, 88, 1058.

5. De Villiers, Wurster, D. E., Van Der Watt, J. G., Ketkar, A., Int. J. Pharm. (Netherlands), 1998, 163, 219.

6. Takeuchi, H., Handa, T., Kawashima, Y., Chem. Pharm. Bull., 1987, 3800.

7. Chiou, W. L., Riegelman, S., J. Pharm. Sci. 1971, 60, 1281.

8. Ford, J. L., Pharm. Acta Helv., 1986, 61, 69.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of coprocessing a limited solubility bioactive agent with a compatible aid comprising the steps of:
(a) identifying a compatible aid for the bioactive agent;
(b) either (i) forming a co-dissolved solution of the compatible aid and bioactive agent in a common solvent or (ii) forming a solution of the compatible aid in an anti-solvent and forming solution of the bioactive agent in a solvent; and
(c) forming a film or primary particles from the solution or solutions of step (b), which film or primary particles comprise bioactive agent in crystalline form, with the crystals having average diameter of 1 micron or less;
wherein the film or particles provide faster dissolution of the bioactive agent, or greater bioactive agent bioavailability, or faster onset.

2. The method of claim 1, wherein the forming of step (c) forms primary particles.

3. The method of claim 1, wherein the forming of step (c) is conducted by
(i) spray drying the co-dissolved solution to remove the solvent, or
(ii) (1) mixing the co-dissolved solution with an antisolvent for the compatible aid and bioactive agent using impinging jets, or
(ii)(2) mixing the bioactive agent solution with the solution of compatible aid in antisolvent using impinging jets, or
(iii) conducting process (ii)(1) or (ii)(2) and drying the product by spray drying, or
(iv) batch precipitation of the co-dissolved solution, or batch precipitation of the solution of bioactive agent with the compatible aid in antisolvent.

4. The method of claim 1, wherein the step of identifying a compatible aid comprises dissolving the bioactive agent and a potential compatible aid in a common solvent, evaporating and precipitating the resultant solution, and analyzing the resultant blend or film for improved dissolution and the presence of at least 20% of the bioactive agent in a crystalline form.

5. The method of claim 1, wherein the bioactive agent is a COX-2 selective inhibitor.

6. The method of claim 1, wherein the compatible aid is a block copolymer of propylene oxide and ethylene oxide.

7. The method of claim 1, wherein the compatible aid is a polymer.

8. The method of claim 1, wherein the compatible aid is a block copolymer of propylene oxide and ethylene oxide.

9. A formulation of a bioactive agent made by the method of claim 1.

10. A formulation of a COX-2 selective inhibitor made by the method of claim 5.

11. A formulation of a COX-2 selective inhibitor made by the method of claim 8.

12. A formulation comprising a crystalline form of COX-2 selective inhibitor co-processed with a water soluble polymer to form primary particles of approximately 15 microns or less.

13. The formulation of claim 12, wherein the polymer is a block copolymer of propylene oxide and ethylene oxide.

14. The formulation of claim 12, wherein the COX-2 selective inhibitor is selected from the group consisting of bisarylheterocyclic compounds.

15. The formulation of claim 12, wherein the COX-2 selective inhibitor is (Z)-3-[1-(4-bromophenyl)-1-(4-methylsulfonylphenyl) methylidene]-dihydrofuran-2-one.

16. The formulation of claim 12, wherein the COX-2 selective inhibitor is (Z)-3-[1-(4-chlorophenyl)-1-(4-methylsulfonylphenyl) methylidene]-dihydrofuran-2-one.

17. The formulation of claim 12, wherein the COX-2 selective inhibitor is (Z)-3-[1-(4-bromophenyl)-1-(4-methylsulfonylphenyl) methylidene]-dihydrofuran-2-one and the polymer is a block copolymer of propylene oxide and ethylene oxide.

18. The formulation of claim 17, wherein the block copolymer of propylene oxide and ethylene oxide is Pluronic™ F127.
